(19) United States
(12) Patent Application Publication
(10) Pub. No.: US 2026/0334979 A1
(43) Pub. Date: Oct. 29, 2026

(54) THORACIC SKELETAL SYSTEM TREATMENT WITH BIOLOGICS

(71) Applicant: ThoraGenix Innovations, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: ThoraGenix Innovations, Inc., Tampa, FL (US)

(21) Appl. No.: 19/390,341

(22) Filed: Nov. 14, 2025

Related U.S. Application Data
(60) Provisional application No. 63/723,441, filed on Nov. 21, 2024.

(51) Int. Cl. A61B 17/70; A61B 17/56; A61B 17/82; A61K 38/18

(52) U.S. Cl. CPC A61B 17/70; A61B 17/823; A61K 38/1875; A61B 2017/564

(57) ABSTRACT

Systems, methods, and devices disclosed herein include a thoracic skeletal structure treatment system for healing bone with a biologics-based bone healing agent. The system(s) are used to perform a method including positioning a biologics-based bone healing agent at a target area of a thoracic skeletal structure. The method also includes securing the biologics-based bone healing agent at the target area using one or more reinforcement structures resulting in secured biologics-based bone healing agent at the target area. The secured biologics-based bone healing agent can form a compressed environment, which causes bone to heal, with the secured biologics-based bone healing agent, at the target area. In this way, the disclosed thoracic skeletal structure treatment system can create bone growth instigation environment with a stabilization environment at the target area.

### CROSS REFERENCE TO RELATED APPLICATION

This application claims priority to U.S. Provisional Patent Application Ser. No. 63/723,441, filed Nov. 21, 2024, and titled “THORACIC SKELETAL SYSTEM TREATMENT WITH BIOLOGICS,” which is incorporated by reference herein in its entirety.

### BACKGROUND

Typical bone healing procedures only focus on stabilization of the damaged bones. This approach has many drawbacks. Merely stabilizing bones does not provide all necessary conditions to promote bone growth. Also, some bone systems have a high degree of articulation, such that stabilization techniques result in reduced mobility. Furthermore, wires, plates, and screws used for stabilization often require a significant compressive force to be effective, yet this compressive force can cut into or otherwise cause damage to the surface of the bone.

It is with these observations in mind, among others, that various aspects of the present disclosure were conceived and developed.

### SUMMARY

Systems, methods, and devices disclosed herein can address the aforementioned problems. For instance, a method of thoracic skeletal structure treatment can include positioning a biologics-based bone healing agent at a target area of a thoracic skeletal structure. The method can also include securing the biologics-based bone healing agent at the target area using one or more reinforcement structures resulting in secured biologics-based bone healing agent at the target area. Moreover, the method can include causing bone to heal, with the secured biologics-based bone healing agent, at the target area.

In some examples, the target area of the thoracic skeletal structure can include at least one of a thoracic cage portion, a rib portion, a sternum portion, a clavicle portion, a cervical vertebrae portion, a thoracic vertebrae portion, a lumbar vertebrae portion, or a scapula portion. Additionally, the target area can include at least one of a damaged bone tissue, a degenerated bone tissue, a demineralized bone tissue, a genetically deformed bone tissue, an idiopathically deformed bone tissue, an infected bone tissue, an iatrogenically destabilized bone tissue, or a traumatized bone tissue. Furthermore, the biologics-based bone healing agent can have a bone growth factor including at least one of exosomes or bone morphogenetic proteins (BPM)s. The biologics-based bone healing agent can also have a form factor including at least one of a bone graft, a synthetic graft, a putty, a gel, a bioglass, a powder, an injectable gel or liquid, or a collagen matrix.

In some scenarios, the method can further include reducing bleeding at the target area using the biologics-based bone healing agent. Furthermore, the target area can include a first sternal section separated from a second sternal section during a sternotomy, and/or the causing of the bone to heal at the target area can include performing a sternal closure and/or reconstruction for the first sternal section and the second sternal section. Additionally, the target area can include a rib fracture, and/or the causing of the bone to heal at the target area can include a rib fixation and healing procedure at the rib fracture. The target area can also include a thoracic spinal vertebra, and/or the causing of the bone to heal at the target area can include a spinal fusion and stabilization procedure at the thoracic spinal vertebra. Moreover, the target area can include a bone void, and/or the positioning of the biologics-based bone healing agent can include filling the bone void with a regenerative bone putty. The method can further include positioning a thoracic skeletal system stabilization hardware device around the target area, and/or the positioning of the biologics-based bone healing agent can include filling a gap between the thoracic skeletal system stabilization hardware device and bone tissue with a regenerative bone putty. The method can also include positioning a thoracic skeletal system stabilization hardware device around the target area, and/or the positioning of the biologics-based bone healing agent can include filling a screw hole of the thoracic skeletal system stabilization hardware device with a regenerative bone putty.

In some instances, a method of thoracic skeletal structure treatment can include positioning a biologics-based bone healing agent at a target area located at one or more of a rib, a sternum, or a thoracic vertebra. The method can also include securing the biologics-based bone healing agent at the target area resulting in secured biologics-based bone healing agent at the target area. Moreover, the method can include causing a damaged portion of bone to heal, with the secured biologics-based bone healing agent, at the target area.

In some examples, the biologics-based bone healing agent includes a sternal implant disposed between two sternal bone sections, and/or the securing of the biologics-based bone healing agent can include compressing the two sternal bone sections together with one or more wires. The one or more wires can include one or more force dispersion supplements which increase a surface area of the one or more wires against the two sternal bone sections. Also, the target area can include a portion of a thoracic spinal column stabilized with stabilization hardware including at least one of: a pedicle screw, a plate, or a wire. Additionally, the positioning of the biologics-based bone healing agent at the target area can include filling a space around and/or adjacent the stabilization hardware. The positioning of the biologics-based bone healing agent at the target area can also include filling a void with the biologics-based bone healing agent resulting in a filled void, and/or the securing of the biologics-based bone healing agent at the target area can include creating a rigid environment under compression at the filled void which instigates bone growth.

In some scenarios, a method of thoracic skeletal structure treatment includes positioning a biologics-based bone healing agent at a target area of a thoracic skeletal structure by filling a bone void, resulting in a filled bone void. The method can also include securing the biologics-based bone healing agent at the target area resulting in secured biologics-based bone healing agent at the target area. Furthermore, the method can include causing bone to heal, with the secured biologics-based bone healing agent, at the target area, for instance, by instigating bone growth with the secured biologics-based bone healing agent. The bone void can be located on at least one of: a rib, a sternum, or a vertebra. Additionally, the biologics-based bone healing agent can include a liquid, gel, or putty, and/or securing the biologics-based bone healing agent can include solidifying (e.g., curing and/or drying) the liquid, the gel, or the putty. Furthermore, the securing of the biologics-based bone healing agent can include mechanically stabilizing the target area and applying compression on the filled bone void, and/or the causing of the bone to heal can include instigating bone growth responsive to the compression on the filled bone void.

### BRIEF DESCRIPTION OF THE DRAWINGS

FIG. 1 illustrates an example system for treating a thoracic skeletal system with a biologics-based bone healing agent.

FIGS. 2A and 2B illustrate an example system for performing a sternal closure and/or reconstruction procedure with a biologics-based bone healing agent, which can form at least a portion of the system depicted in FIG. 1.

FIGS. 3A and 3B illustrate an example system for performing a rib fixation and healing procedure with a biologics-based bone healing agent, which can form at least a portion of the system depicted in FIG. 1.

FIG. 4 illustrates an example system for treating various types of target areas of a thoracic skeletal system with a biologics-based bone healing agent, which can form at least a portion of the system depicted in FIG. 1.

FIG. 5 illustrates an example system for performing a spinal fusion and stabilization procedure with a biologics-based bone healing agent, which can form at least a portion of the system depicted in FIG. 1.

FIG. 6 illustrates an example system for treating a thoracic skeletal system with a biologics-based bone healing agent, which can form at least a portion of the system depicted in FIG. 1.

FIG. 7 illustrates an example method of treating a thoracic skeletal system with a biologics-based bone healing agent, which can be performed by any of the systems depicted in FIGS. 1-6.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein can heal bones of the thoracic skeletal system using biologics. These techniques can be used for sternal closure and reconstruction procedures, rib fracture fixation and healing procedures, and/or thoracic spinal fusion and stabilization procedures. The use of biologics in these bone healing procedures can promote bone growth in addition to providing adequate bone stabilization by filling space and providing a medium into which the bone can grow. The biologics can create an environment at a damaged portion of the bone which instigates growth of new bone. This can include filling voids in the damaged bone to bridge a bone growth trajectory gap which would otherwise prevent bone growth. Moreover, filling the bone voids can provide a volume of material with which a compressive environment can be created and sustained, further instigating bone growth.

Application of these techniques can benefit the thoracic skeletal system due to the wide array of articulation points in the thoracic skeletal system. The sternum operates as a first anchor point for the articulation of the plurality of ribs connecting to the sternum, and the thoracic spinal column operates as a second anchor point. The thoracic skeletal system articulates during many locomotive actions which can involve bending or twisting the thoracic spinal column. Even breathing itself requires the articulatable motion of the ribs. The technology disclosed herein can improve thoracic bone healing techniques by focusing on both stabilization and bone growth such that natural bone growth rebuilds damaged areas of the thoracic skeletal system and restores the full range of motion. This is a marked improvement over other techniques which focus solely on stabilization and result in reduced range of motion and limited mobility.

Additional benefits and advantages of the disclosed technology will become apparent from the detailed description below.

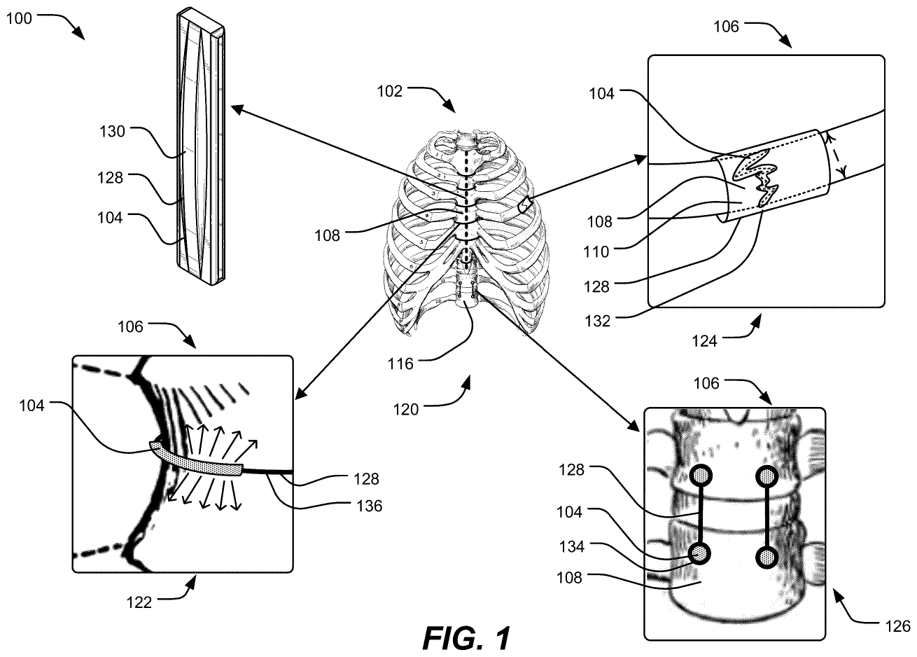

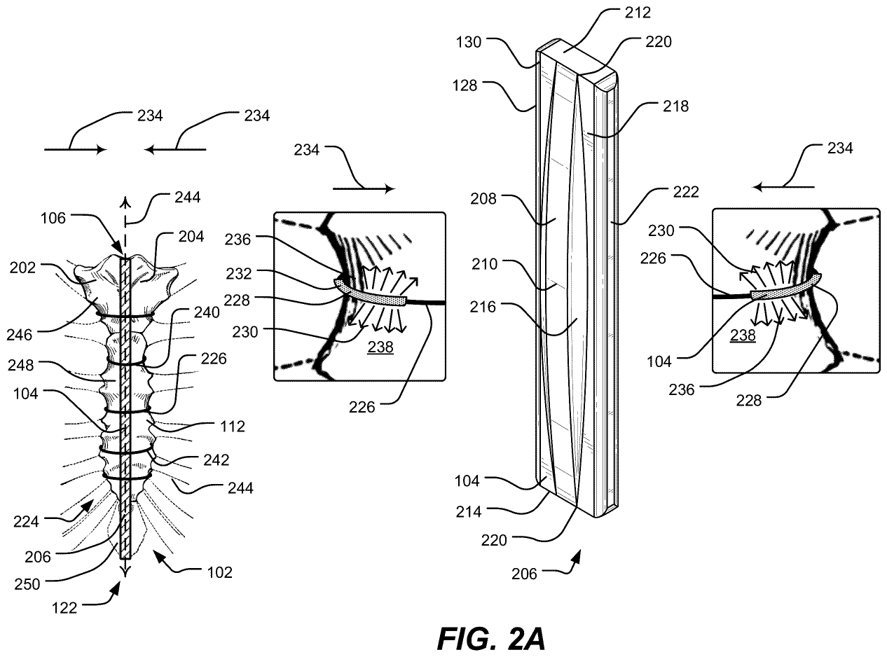

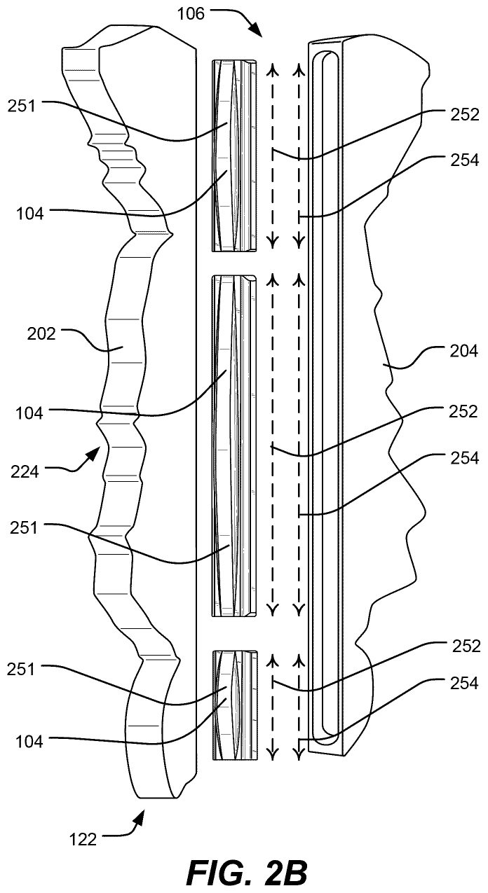

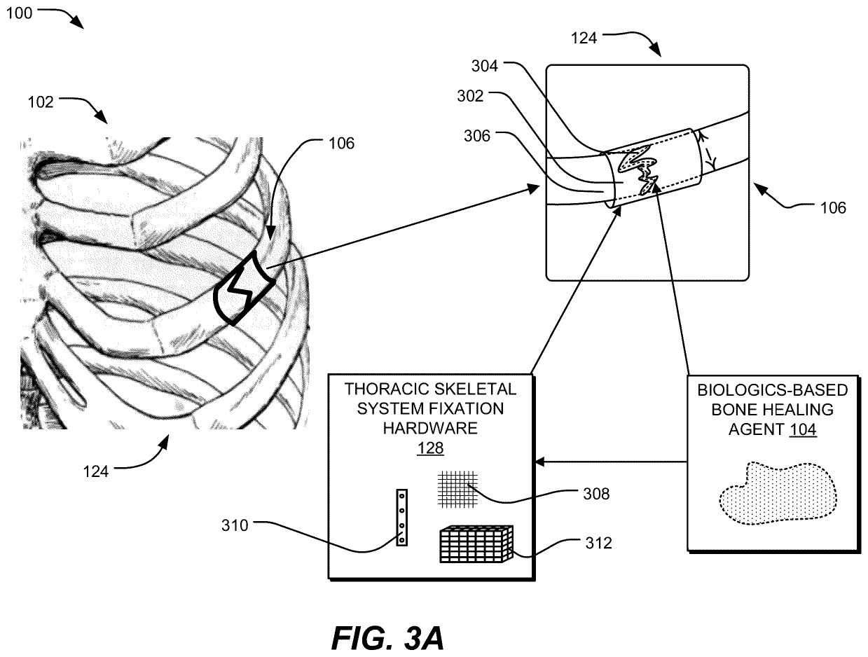

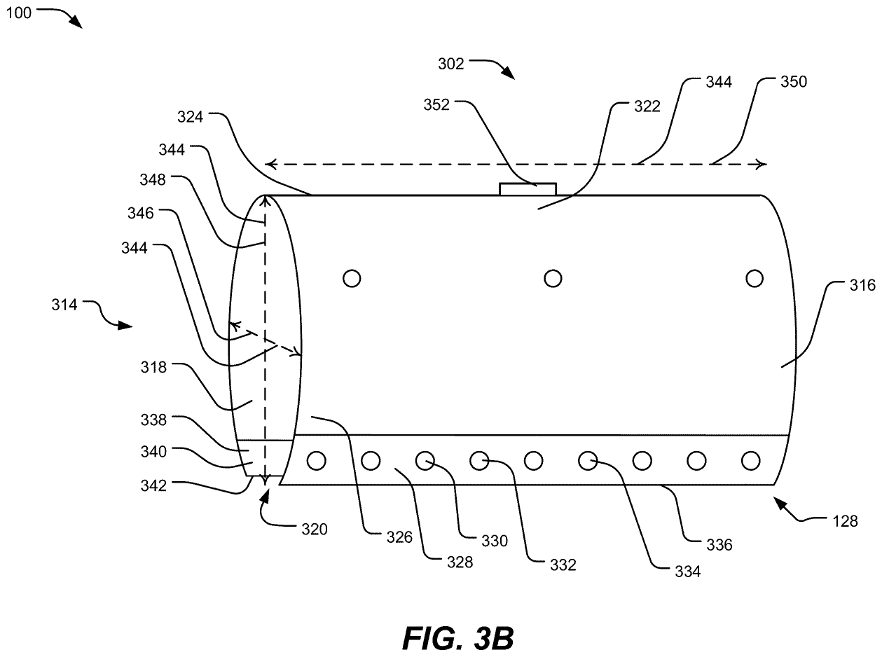

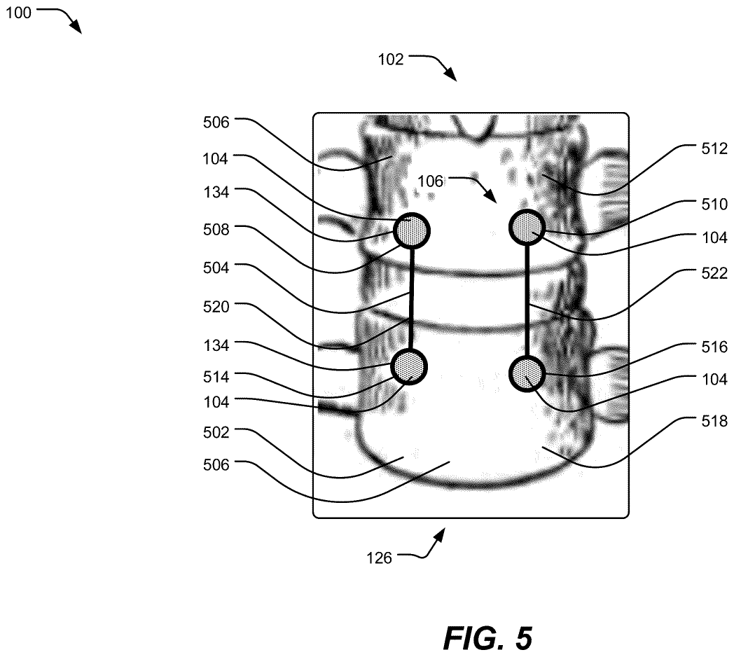

FIG. 1 illustrates an example system 100 for treating a thoracic skeletal system 102 by introducing a biologics-based bone healing agent 104 into one or more target area(s) 106 of a portion of bone 108 being treated. The target area 106 can include any portion of the thoracic skeletal cage, such as a rib portion 110, a sternum portion 112, a clavicle portion, a vertebra portion 114 (e.g., a thoracic vertebra 116), and/or a scapula portion. For instance, a bone healing treatment procedure 120 can include a sternal closure and/or reconstruction procedure 122 in which the target area(s) 106 include one or more sternum portion(s) 112, such as a first sternal section separated from a second sternal section during a sternotomy (e.g., as shown in FIGS. 2A and 2B). Additionally or alternatively, the bone healing treatment procedure 120 can include a rib fixation and healing procedure 124 at the rib portion 110 (e.g., as shown in FIGS. 3A and 3B). Furthermore, the bone healing treatment procedure 120 can include a spinal fusion and stabilization procedure 126 at the vertebra portion 114 (e.g., as shown in FIG. 5).

In some examples, the bone healing treatment procedure 120 can include application of the biologics-based bone healing agent 104 to the target area(s) 106 to instigate bone growth. The biologics-based bone healing agent 104 can have a material composition (e.g., a viscosity and/or rigidity) to hold itself in place at the target area(s) 106, and/or an additional thoracic skeletal system fixation hardware 128 can be used to maintain the biologics-based bone healing agent 104 in place. Furthermore, the biologics-based bone healing agent 104 can be used to augment other thoracic skeletal system fixation hardware 128, such as a sternal implant 130, a rib fixation device 132, one or more pedicle screws 134, a fixation wire 136, and/or other types of plates, wires, and/or screws.

In some instances, the biologics-based bone healing agent 104 can include a bone graft, a synthetic graft, a regenerative bone growth putty, a regenerative bone growth gel or liquid, bone morphogenetic proteins (BMP)s, exosomes, bioglass, a powder, an injectable material, a collagen matrix, any combinations thereof, and/or any other bone growth factors. In some scenarios, the biologics-based bone healing agent 104 can comprise a bioglass including a bioactive ceramic material, such as a material including P₂O₅, SiO₂, CaO, Na₂O and CaO. Moreover, the biologics-based bone healing agent 104 can include a regenerative putty with a particular composition to optimize the bone healing treatment procedure 120. For instance, the regenerative putty can be formed of a matrix material, such as demineralized bone matrix (DBM) and/or a collagen matrix (e.g., a bovine collagen matrix), and/or the bioglass. These components can make the regenerative putty conductive and/or inductive such that cells (e.g., blood cells) are drawn into the regenerative putty. Moreover, the composition of the regenerative putty may be irrigation-resistant with respect to blood. The regenerative putty can include various bone growth compounds (e.g., osteoblasts, stem cells, exosomes, and so forth).

FIGS. 2A and 2B illustrate an example system 100 for treating the thoracic skeletal system 102 with the biologics-based bone healing agent 104. As depicted in FIGS. 2A and 2B, the one or more target area(s) 106 can include one or more sternum portion(s) 112, such as a first sternal section 202 separated from a second sternal section 204 during a sternotomy. The system(s) 100 depicted in FIGS. 2A and 2B can be similar to, identical to, and/or can form at least a portion of the system(s) 100 discussed above regarding FIG. 1.

In some examples, the biologics-based bone healing agent 104 can include a sternal graft 206 (e.g., implant) operable for placement between the first sternal section 202 and the second sternal section 204. The sternal graft 206 can include a substrate material which is soaked and/or coated in the biologics-based bone healing agent 104, and/or the structural material itself of the sternal graft 206 may be formed of the biologics-based bone healing agent 104. Additionally, the sternal graft 206 can include a geometric profile to optimize implantation of the biologics-based bone healing agent 104 into the target area 106. This can include a first fin 208 extending with a curved depth, such that the depth value (e.g., in a z-axis direction) gradually changes to form a curved side profile. The curved side profile can have a greatest depth value at a y-axis midpoint 210. Additionally or alternatively, the greatest depth value can be at a location between the y-axis midpoint 210 and a first end 212 of the sternal graft 206 (e.g., a top end), and/or a second end 214 of the sternal graft 206 (e.g., a bottom end). In other words, the greatest depth value can be positioned anywhere along the y-axis (e.g., at eighths, sevenths, fifths, quarters, thirds, and/or a halfway point).

In some instances, the first fin 208 can have a tapered width 216 such that a width value (e.g., in an x-axis direction) gradually changes from the first end 212 to the second end 214, thus forming a curved front profile. The curved front profile can include rounded, chamfered corners between the first fin 208 and a central body portion 218 from which the first fin 208 extends. These rounded, chamfered corners can have a tapered width such that they terminate at points 220 at the first end 212 and/or points 220 the second end 214. The first fin 208 can have many different shapes and/or sizes to provide the functionality disclosed herein (e.g., different depth values, different width values, different lengths, flat or curved) and/or can extend only a portion of the central body portion 218.

Furthermore, in some examples, the central body portion 218 can include a tapered front 222 and/or a tapered back. The tapered front 222 and/or the tapered back can include a tapering depth value (e.g., in the x-direction) such that the leading edges of the sternal graft 206 have generally elongated trapezoidal prism-shaped profiles. In other words, the front surface and/or the back surface of the sternal graft 206 can have narrower depth values and/or lesser length values than the central body portion 212.

In some scenarios, the sternal graft 206 can include a second fin, extending from a second sidewall of the central body portion 218, which can be similar to and/or identical to the first fin 208, such that the first fin 208 and/or the second fin give the sternal graft 206 a symmetrical profile. Additionally or alternatively, the second fin can have a different shape than the first fin 208, such that their respective greatest depth value(s) are different and/or at different locations in the y-axis direction. The fins can also have different tapered width dimension(s). Furthermore, the first fin 208 and/or the second fin can have curved side profile lengths which extend from the first end 212 to the second end 214 (e.g., a full length of the sternal graft 206 in the y-axis direction). Moreover, in some examples, the first fin 208 and/or the second fin can extend only or at least 90%, 80%, 70%, 60%, 50%, 40%, 30%, 20% or 10% of the length of the sternal graft 206 in the y-axis direction. Some example sternal grafts 206 can include a single first fin 208 extending from the first sidewall and/or a single second fin extending from the second sidewall 160. These singular fins can be centrally positioned along the length of the sternal graft 206 (e.g., with the location of the greatest depth value at the y-axis midpoint 210), and/or can extend a majority and/or an entire length of the sternal graft 206. Also, rather than singular fins, the first fin 208 and/or the second fin can include a plurality of fins forming one or more rows of fins along the first sidewall and/or the second side wall.

In some examples a particular fin profile shape feature, such as the location of the greatest depth value and/or the various tapers and curves disclosed herein, can correspond to a particular bone shape of a target bone being healed with the sternal graft 206. For instance, the location of the greatest depth value can correspond to a wider or deeper portion of the target bone, such as a particular location on the sternum 224. In this way, the curved side profile can cause the sternal graft 206 to be especially effective at delivering the biologics-based bone healing agent 104 to the interior of the bone while securing to and healing the bone. By way of example, the sternal graft 206 can have a width dimension of between 0.8 cm and 1.8 cm (e.g., 1.2 cm, 1.3 cm, 1.4 cm, etc.). The sternal graft 206 can have a depth dimension of between 0.4 cm and 1.2 cm (e.g., 0.7 cm, 0.8 cm, 0.9 cm etc.). Furthermore, the sternal graft 206 can have a length dimension of between 5.0 cm and 9.0 cm (e.g., 6.0 cm, 7.0 cm, 8.0 cm, etc.) As such, the sternal graft 206 can have a width dimension, a depth dimension, and/or a length dimension sized for placement between the first sternal section 202 and the second sternal section (e.g., a left half of the sternum 224 and the right half of the sternum 224).

In some instances, the sternal graft 206 can be formed of the biologics-based bone healing agent 104 including at least one of a DBM, cortical bone fibers, cancellous bone fibers, collagen sponge, cortical bone graft, synthetic bone, tissue graft, and/or combinations thereof. For instance, the sternal graft 206 can include a collagen matrix derived from bovine, porcine, plant fibers, and/or any combination thereof. The collagen matrix can be embedded in a center of the sternal graft 206 (e.g., forming the central body portion 218) and/or the collagen matrix can form the first fin 208 and/or the second fin. Moreover, this collagen lattice matrix can be designed to provide additional structural integrity to the body of the sternal graft 206, giving the sternal graft 206 increased strength and structural integrity when sheer forces and other natural thoracic skeletal system movement forces interact with the sternal graft 206 after it has been implanted.

In some scenarios, the sternal graft 206 can also include the biologics-based bone healing agent 104 as one or more carrier agents, such as a cellular bone growth factor, antibiotics, analgesics, medications, adhesives, delivery vehicles, and/or combinations thereof. For instance, the bone growth factor can include bone morphogenetic proteins, mesenchymal stem cells, blood, osteoclasts, osteoblasts, hydroxyapatite (HA), exosomes, and/or combinations thereof. The adhesives can include a mollusk-based adhesive. The delivery vehicles can include one or more of bioglass, ceramics, and/or ceramics-derivatives. The methods disclosed herein can further include soaking the implant in the at least one carrier agent (e.g., the cellular growth factor) during the surgical procedure and/or prior to insertion into the bone sections. Additionally, the matrix of the sternal graft 206 can have a similar composition and/or structure as surgical mesh or gauze, such as an intertwined fiber crosshatch.

In some examples, the system(s) 100 can include thoracic skeletal system fixation hardware 128, such as one or more fixation wire(s) 226 (e.g., similar or identical to the fixation wire(s) 136 in FIG. 1) wrapped around the first sternal section 202 and the second sternal section 204. The fixation wire(s) 226 can include one or more force dispersion supplement(s) 228, which can comprise the biologics-based bone healing agent 104. The force dispersion supplement(s) 228 can be disposed on the fixation wire(s) 226 to increase a force dispersion surface area 230 of the fixation wire(s) 226 which optimizes a placement, securement, and/or a healing step of the sternal closure and/or reconstruction procedure 122. The force dispersion supplement(s) 228 can include material formed onto at least a portion 232 of the fixation wire 226. The force dispersion supplement(s) 228 can include a different material than the hard metal of the fixation wire 226, such as a polymer material, silicon, a fibrous mesh material, and/or a softer type of metal material. In some examples, the force dispersion supplement 228 can include a coating of the biologics-based bone healing agent 104, a silicon coating, and/or combinations thereof. The force dispersion supplement(s) 228 can also be formed of a same material as the fixation wire 226.

In some instances, the force dispersion supplement(s) 228 can increase the force dispersion surface area 230 of the sternal fixation device 102, for instance, by increasing a diameter dimension and/or a width dimension of the fixation wire 226. As a compressive force 234 is applied with the fixation wire 226, the force dispersion supplement(s) 228 can be pressed against the first sternal section 202 and/or the second sternal section 204 at one or more attachment zones 236. This can cause the force dispersion supplement(s) 228 to flatten and/or expand (e.g., in scenarios involving flexible/elastic force dispersion supplement(s) 228), which can further increase the force dispersion surface area 230 at the attachment zone(s) 236. As such, the fixation wire 226 can have increased surface contact with the attachment zones 236, which decreases the amount of force experienced at any individual contact point of the attachment zones 236. Accordingly, the surgeon can eliminate gapping and/or loosening of the fixation wires 226 by providing an adequate compressive force 234 with the fixation wire 226 to secure the first sternal section 202 and the second sternal section 204 together for proper healing, while decreasing an amount of damage caused to the bone tissue 238 by the fixation wire(s) 226 (e.g., sawing and/or cutting into the bone tissue 238).

In some examples, the thoracic skeletal system fixation hardware 128 can include a plurality of fixation wires 240. Individual fixation wires 226 of the plurality of sternal fixation wires 240 can correspond to different anatomical locations of the target bone, for instance, by having particular length dimensions, width dimensions, material hardnesses, or so forth, which matches the dimensions, contours, and/or bone density at the particular target locations. The plurality of fixation wires 240 can correspond to and/or be operable for placement at a plurality of attachment zone(s) 242. The plurality of attachment zones 242 can be located between different costal notches for a plurality of ribs 244. These attachment zones 242 can be located across a center midline 244 from each other, the center midline 244 dividing the first sternal section 202 and the second sternal section 204. The plurality of attachment zone(s) 242 can include lateral borders and/or outer sternal edges at the manubrium 246 (e.g. between an articular facet for rib 1 and an articular demifacet for rib 2), the sternum body 248, and/or the xiphoid process 250. The compressive force 234 applied by the plurality of fixation wires 240 can push, pull, squeeze, bias, and/or compress the first sternal section 202 and/or the second sternal section 204 toward the center midline 244 by distributing the compressive force 234 at the plurality of attachment zone(s) 242.

In some examples, the force dispersion supplement(s) 228 can cover or conceal a full circumferential surface of at least a portion of the fixation wire 226. The fixation wire 226 can include one or more bare portion(s) which omit the force dispersion supplement(s) 228 and/or one or more supplemented portion(s) which include the force dispersion supplement(s) 228 covering or concealing the full circumferential surface of the supplemented portion. The force dispersion supplement 228 can be a sleeve permanently affixed to the fixation wire 226 or slidable along the fixation wire 226. The supplemented portion(s) of the fixation wire 226 can have the force dispersion supplement 228 wrapped fully around the circumference of the fixation wire 226. Accordingly, the supplemented portion(s) can have a first diameter dimension which is expanded and/or greater than a second diameter dimension of the bare portion(s), which increases the force dispersion area 230. Furthermore, the supplemented portion(s) can be selectively arranged along a length of the fixation wire 226 such that the supplemented portion(s) align with an outer sternal edge and/or other high-pressure contact points of the first sternal section 202 and/or the second sternal section 204. In some instances, the force dispersion supplement 228 can cover or conceal an entire length of the fixation wire 226 so that the fixation wire 226 does not include bare portion(s), and/or is fully coated with the biologics-based bone healing agent 104. It is to be understood that the various configurations disclosed herein can be selected by the surgeon based on the particular anatomical structures of the bones being healed together. For instance, the system 100 can include a plurality of fixation wires 240 having these different configurations, for instance in a group or pack. Moreover, other types of bone sections beyond the sternum 224 can be adjoined and healed using the technology disclosed herein.

Furthermore, in some examples, the force dispersion supplement(s) 228, and/or the biologics-based bone healing agent 104, can only cover or conceal a side of the fixation wire 226 at the supplemented portion(s). For instance, a concealed or covered side of the supplemented portion can include the force dispersion supplement 228 and a bare side of the supplemented portion can omit the force dispersion supplement. In this configuration, the force dispersion supplement 228 can cover or conceal less than 100% of the circumference of the fixation wire 226, such that the circumference of the supplemented portion is only partially concealed or partially covered by the force dispersion supplement 228. The covered side can be directed towards the attachment zone(s) 242 (e.g., during an orientation operation performed by the surgeon), while the bare side can be directed away from the attachment zone(s) 242. The covered side can be an interior side while the bare side can be an exterior side of the fixation wire 226. Also, the covered side can be an opposite side from the bare side, for instance, located across the first diameter from the bare side. In some instances, the force dispersion supplement 228 and/or the biologics-based bone healing agent 104 can include a partial or half sleeve, or a pad, such as a mesh, interwoven, and/or fibrous pad disposed between the attachment zone(s) 242 and the fixation wire 226.

In some examples, the techniques disclosed herein can be used for bone repair, for instance, to heal damage caused by wires, plates and/or other fixation hardware 128. For instance, the fixation wires 226 used in combination with the sternal graft 206 can cause stress risers, stress fractures, and/or other damaged portions due to the friction and/or pressure of the fixation wire 226 against the bone. Additionally or alternatively one or more plates and/or struts can be used (e.g., in combination with or alternatively to the fixation wires 226) to secure the first sternal section 202 to the second sternal section 204. Stress points where the plates and/or struts contact the bone can also cause bone damage. This bone damage can be repaired with placement of biologics-based bone healing agent 104 at the damaged areas and/or stress areas, which can cause bone growth at the damaged and/or stressed area.

Turning to FIG. 2B, an example system 100 for treating a thoracic skeletal system 102 with a biologics-based bone healing agent 104 is depicted. For instance, the system 100 can include a plurality of sternal graft segments 251 including the biologics-based bone healing agents 104 disposed between the first sternal section 202 and the second sternal section 204 during the sternal closure and/or reconstruction procedure 122. In some scenarios, one or more sternal graft segments 251 can have one or more first length dimension(s) 252 which corresponds to one or more second length dimension(s) 254 of different portions of the sternum 224. In other words, the plurality of sternal graft segments 251 can have different first length dimensions 252 corresponding to different second length dimensions 254 based on the anatomy of the bone sections being fused together. For instance, a first bone healing implant 256 can have a first length dimension 252 corresponding to a dimension of the manubrium 246 for placement between two sections of the manubrium 246. A second bone healing implant 258 can have a first length dimension 252 corresponding to a dimension of a sternum body 248 for placement between two sections of the sternum body 248. Additionally or alternatively, a third bone healing implant 260 can have a first length dimension 248 corresponding to a dimension of the xiphoid process 250 for placement between two sections of the xiphoid process 250.

FIGS. 3A and 3B illustrate an example system 100 for treating the thoracic skeletal system 102 with the biologics-based bone healing agent 104. As depicted in FIGS. 3A and 3B, the one or more target area(s) 106 can include one or more rib portion(s) 110 including a rib fracture. The system(s) 100 depicted in FIGS. 3A and 3B can be similar to, identical to, and/or can form at least a portion of the system(s) 100 discussed above regarding FIG. 1 In some scenarios, the thoracic skeletal system fixation hardware 128 can include one or more (e.g., a plurality) of various structures used for positioning and/or securing the biologics-based bone healing agent 104 at a target area 106. The thoracic skeletal system fixation hardware 128 can include a rib fixation device 302 (e.g., similar or identical to the rib fixation device 132 of FIG. 1) with a particular form factor designed to fit around a damaged portion 304 of a rib 306. Additionally or alternatively the thoracic skeletal system fixation hardware 128 can include a fiber mesh 308 which can be applied to the damaged portion 304. Then, a strut 310, a cage 312, and/or the rib fixation device 302 can be placed over the fiber mesh 308. With the fiber mesh 308, the strut 310, the cage 312, and/or the rib fixation device 302 in place, open space within the fiber mesh 308 can be filled with the biologics-based bone healing agent 104. Additionally, the underlying structural material of the fiber mesh 308, the strut 310, the cage 312, and/or the rib fixation device 302 can be formed of the biologics-based bone healing agents 104 disclosed herein.

In some examples, a layer of the biologics-based bone healing agent 104 can be applied to the target area 106 prior to application of the fiber mesh 308, the strut 310, the cage 312, and/or the rib fixation device 302. For instance, a method of using the system 100 can include applying a first layer of the biologics-based bone healing agent 104 before applying the thoracic skeletal system fixation hardware 128 and applying a second layer of the biologics-based bone healing agent 104 after applying the thoracic skeletal system fixation hardware 128. In some scenarios, the bone healing process can include positioning the fiber mesh 308 under and/or adjacent to the strut 310, and/or filling space within and around the fiber mesh 308 and the strut 310 with the biologics-based bone healing agent 104, such as gaps, screw holes, or so forth. Additionally or alternatively, the thoracic skeletal system fixation hardware 128 can include the cage 310 and/or the rib fixation device 302, which can be positioned around the target area 106 and can have open spaces which can become filled with the biologics-based bone healing agent 104. This bone healing process can include using any one of or any combination of the fiber mesh 308, the strut 310, the cage 312, and/or the rib fixation device 302. The thoracic skeletal system fixation hardware 128 can also include an interbody cage which can be a cylindrical, crescent-shaped, hollow, and/or porous metallic device (e.g., a BAK cage).

The thoracic skeletal system fixation hardware 128 can form an open lattice structure at least partly surrounding the target area 106 and/or integrating into the target area 106. A size, shape, and/or various dimensions of the open lattice structure can correspond to a size, shape, and/or dimension(s) of the target area 106 (e.g., an exposed bone side, a fracture length, or so forth). The open lattice structure formed by the thoracic skeletal system fixation hardware 128 can have an irregular shape formed to correspond to a complex bone defect being healed by the system 100.

In some examples, the system 100 can be non-load bearing or only partially-load bearing. For instance, the biologics-based bone healing agent 104 can operate to fill space (e.g., within the thoracic skeletal system fixation hardware 128 and/or the target area 106) such that bone growth cells from the surrounding area are pulled into the filled space. By filling the space with the biologics-based bone healing agent 104, the system 100 can use the thoracic skeletal system fixation hardware 128 around the space to create a compressed environment which instigates bone growth. Additionally or alternatively, the thoracic skeletal system fixation hardware 128 can provide some structural stabilization and/or load bearing support for the thoracic skeletal system 102 (e.g., by preventing bone movement/sliding).

In some instances, the thoracic skeletal system fixation hardware 128 can include a rigid material such as titanium, polyether etherketone (PEEK), and/or biologics. In some scenarios, the thoracic skeletal system fixation hardware 128 can be formed of a material that is transparent to x-rays and/or other imaging technology. Additionally or alternatively, the thoracic skeletal system fixation hardware 128 can be formed of a flexible material, such as the fiber mesh 308, and/or the thoracic skeletal system fixation hardware 128 can be formed of the biologics-based bone healing agent 104.

Turning to FIG. 3B, the system 100 for treating a thoracic skeletal system 102 with the biologics-based bone healing agent 104 can use the rib fixation device 302 as the thoracic skeletal system fixation hardware 128.

In some instances, the rib fixation device 302 is operable for placement around the damaged portion 304 of the target rib 306 and/or other portion(s) of the thoracic skeletal system 102. The rib fixation device 302 can include an elongated body 314 having a first sidewall 316 separable from a second sidewall 318 to form a gap 320. During a placement procedure, the target rib 306 can be slid through the gap 320 such that the rib fixation devices 302 is positioned at a location corresponding to the damaged portion 304.

In some examples, the rib fixation device 302 can include a hinge 322 formed along a top portion 324 of the elongated body 314. The hinge 322 can be a living hinge formed by the material comprising the elongated body 314. For instance, the elongated body 314 and/or at least the top portion 324 of the elongated body 314 can have a thickness value such that the top portion 324 is flexible and/or bendable. By flexing/bending at the top portion 324, the first sidewall 316 can be separable from the second sidewall 318 to form and/or increase a size of the gap 320 at a bottom portion 326 of the elongated body 314, so that the elongated body 314 can be slid over the target rib 306. Additionally, once the rib fixation device 302 is in place over the target rib 306, the living hinge 322 can be used to bring the first sidewall 316 and the second sidewall 318 back together, closing and/or reducing the gap 320.

In some examples, the rib fixation device 302 can include a screw plate 328 formed into the elongated body 314, for instance, at the bottom portion 326. The screw plate 328 can include one or more screw holes 330, such as a plurality of screw holes 332 arranged in a row 334. The plurality of screw holes 332 can be disposed adjacent to a first bottom edge 336 of the first sidewall 316 and/or the second sidewall 318. The screw plate 328 can include an additional layer of material formed into and/or layered onto the material which forms the elongated body 314. Additionally or alternatively, the screw plate 328 can be at the bottom portion 326 of the first sidewall 316 and/or the second sidewall 318 without any additional material. In other words, the plurality of screw holes 332 can be formed directly into the first sidewall 316 and/or the second sidewall 318. Moreover, the one or more screw holes 330 can be formed at other locations on the elongated body 314, such as anywhere along the first sidewall 316 and/or the second sidewall 318, at the top portion 324, at combinations thereof, and so forth. The systems 100 disclosed herein can include filling the plurality of screw holes 332 with the biologics-based bone healing agent 104 (e.g., the regenerative bone growth putty) before and/or after placement of the rib fixation device 302 around the damaged portion 304.

In some scenarios, the rib fixation device 302 can include a base plate 338, which can be formed into the first sidewall 316 and/or the second sidewall 318. The base plate 338 can include an additional layer 340 of material designed to receive an end of one or more screws passing through the one or more screw hole(s) 330. For instance, the base plate 338 can be disposed adjacent to a second bottom edge 342 of the first sidewall 316 and/or the second sidewall 318 opposite from the screw plate 328, that is, across the gap 320 from the screw plate 328. During a securement procedure, the one or more screw holes(s) 330 can be aligned with the base plate 338 (e.g., and/or can overlap with the base plate 338), and one or more screws, such as a plurality of screws, can be threaded through the screw hole(s) 330. The base plate 338 can operate as a stop or obstruction for the ends of the screws to prevent the screws from passing through the second sidewall 318. The base plate 338 can define a termination point and/or maximum depth of the screws by receiving and stopping the ends of the screws. In this way, the base plate 338 can operate to prevent unintentional puncturing of nearby tissue with the screws. Furthermore, the base plate 338 can be coated with the biologics-based bone healing agent 104 before and/or after receiving the ends of the screws, and/or a space adjacent to the base plate 338 can be filled with the biologics-based bone healing agent 104.

In some examples, the elongated body 314 can have one or more dimensions 344 which correspond to one or more anatomical dimension(s) of the target rib 306. For instance, the elongated body 314 can have a width dimension value 346 in a closed and/or implanted configuration, which corresponds to a width dimension value (e.g., a thickness) of the target rib 306. This correspondence can include the elongated body's width dimension value 346 being a same value as the width dimension value of the target rib 306, or being slightly larger (e.g., 0.1-5 mm larger and/or 0.1%-10% larger). Additionally, the elongated body 314 can have a height dimension value 348 which can correspond to a height dimension value of the target rib 306 (e.g., by being 0.1-5 mm larger and/or 0.1%-10% larger). Moreover, the elongated body 314 can have a length dimension value 350 which can correspond to a first length dimension of the damaged portion 304 of the target rib 306 (e.g., 0.1-5 mm larger and/or 0.1%-10% larger).

In some instances, the length dimension value 350 of the elongated body 314 can be provided such that the elongated body 314 covers both the first length dimension of the damaged portion 304 (e.g., a fractured portion) of the target rib 306 and a second length dimension of an undamaged portion (e.g., an unfractured portion) of the target rib 306. In some scenarios, a ratio of the second length dimension covered by the elongated body 314 to the first length dimension can vary based on a type and/or severity of the damaged portion 304, and a surgeon's discretion in how much stabilization support is needed by extending the elongated body 314 beyond the first length dimension of the damaged portion 304.

Moreover, one or more of the dimension(s) 344 can vary from one end of the elongated body 314 to the other end. This can include a varying width dimension value 346 and/or a varying height dimension value 348, such that the elongated body 314 at least partly tapers from one end to the other. Additionally or alternatively, the surgeon can strategically place the rib fixation device 302 on the target rib 306 at a thicker portion and/or a stronger portion of the target rib 306 to increase the stability and/or structural support provided by the rib fixation device 302. Furthermore, one or more of the dimension(s) 344 can be adjustable prior to performing the placement procedure. For example, the elongated body 314 can have an adjustable length dimension value 350 via one or more segmentation and/or divider lines formed into the elongated body 314. The segmentation and/or divider line(s) can extend vertically along the first sidewall 316 and/or the second sidewall 318 and can include markings, perforations, and/or less/weakened material such that the elongated body 314 can be broken apart at the segmentation and/or divider line(s) to reduce the length dimension value 350. Additionally or alternatively, the elongated body 314 can have telescoping segments (e.g., with different width dimension value(s) 346 and/or height dimension value(s) 348), such that the length dimension value 350 can be increased and/or decreased by a surgeon to match a particular dimension of the target rib 306 and/or the damaged portion 304 of the target rib 306. Also, the first sidewall 316 and/or the second sidewall 318 can include curves and/or contours that match the particular shape(s) and/or contour(s) of different portions of the target rib 306 receiving the rib fixation device 302.

Additionally, in some scenarios, the rib fixation device 302 can undergo one or more reorientation operations subsequent to the placement procedure onto the target rib 306. The rib fixation device 302 can have multiple adjustment degrees of freedom on the target rib 306. For instance, the elongated body 314 can be rotatably adjustable by rotating the elongated body 314 about a center axis which runs along the length dimension value 350. Moreover, the elongated body 314 can be transversely adjustable by sliding in a direction corresponding to the length dimension value 350. Additionally, the elongated body 314 can be tiltably adjustable in multiple dimensions by tilting one end of the elongated body 314 higher or lower (e.g., in the direction of the height dimension value 348) than another end of the elongated body 314, and/or by titling the one end closer or further from a posterior of the patient (e.g., in the direction of the width dimension value 236) than the other end.

In some examples, the thoracic skeletal system fixation hardware 128 (e.g., the rib fixation device 302) can include a tightening mechanism 352 which can be used to bring a first portion of the elongated body 314 (e.g., including the first sidewall 316) together with a second portion (e.g., including the second sidewall 318). For instance, the tightening mechanism 352 can include a threaded screw extending from the top portion 324 of the elongated body 314, disposed between and engaging the two portions. Actuating the tightening mechanism 352 can bring the two portions together to close the gap 320. Additionally or alternatively, actuating the tightening mechanism 352 can be used to secure (e.g., lock) the first portion in place with respect to the second portion. In this way, the tightening mechanism 352 can operate as a locking mechanism. That said, in some instances, the rib fixation device 302 can include a locking mechanism which is separate from the tightening mechanism 352, such as a tab, locking screw, or bolt. An actuation of the tightening mechanism (e.g., in another direction), can also be used to release the first portion 306 from the second portion 308 and/or increase a dimension of the gap 320. Additionally, the tightening mechanism 352 and/or the locking mechanism can be coated with the biologics-based bone healing agent 104.

Furthermore, the thoracic skeletal system fixation hardware 128 (e.g., the rib fixation device 302) can include one or more portals 354 formed into the first sidewall 316, second sidewall 318, and/or other portion(s) of the thoracic skeletal system fixation hardware 128. The portal(s) 354 can include openings, apertures, luer injection ports, or so forth which, provide a pathway from an exterior of the elongated body 314 to an interior of the elongated body 314. In some instances, the portal(s) 354 can be multi-functional. For example, the portal(s) 354 can be operable to receive an injection of the biologics-based bone healing agent 104 (e.g., the regenerative bone growth putty) once the thoracic skeletal system fixation hardware 128 is positioned over the damaged portion 304. The portal(s) 354 can also be operable to receive bicortical screws, which can be inserted into the portal(s) 354 to further secure the thoracic skeletal system fixation hardware 128 onto the target area 106. The portal(s) 354 can include threading for receiving the bicortical screw(s). Moreover, the portal(s) 354 can include a transparent material and/or cover to form a window which provides visual access to the interior of the elongated body 314. In some instances, the thoracic skeletal system fixation hardware 128 can include any number of portal(s) 354 such as one, two, three, four, five, six, seven, eight, or so forth. The portal(s) 354 can be circular, oval, square, rectangular, triangular, and/or an irregular shape. Additionally, the portal(s) 354 can include removable covers, which can be placed over the portal(s) 354 before and/or after use.

In some instances, the first sidewall 316 can mate with the second sidewall 318 when the rib fixation device 302 is in a closed configuration. The first bottom edge 336 of the first sidewall 316 can abut and/or can overlap with the second bottom edge 342 of the second sidewall 318 in the closed configuration, such that a dimension of the gap 320 is reduced to zero. The first bottom edge 336 and/or the second bottom edge 342 can be linear. Alternatively, the first bottom edge 336 and/or the second bottom edge 342 can have a wave profile edge, a zig-zag profile, a square step profile, or so forth. Additionally, the bottom portion 326 of the elongated body 314 (e.g., and/or other portion(s) such as a side portion) can include one or more channels defined into the material to provide additional space for blood vessels at the target rib 306. The channel(s) can include an elongated indentation or additional extension of the material which runs along the bottom portion 326 of the elongated body 314 in the direction of the length dimension value 350. Additionally or alternatively, the channel(s) can include one or more cutouts or openings to provide space for the blood vessels.

In some examples, the rib fixation device 302 can have a consistent thickness value throughout the elongated body 314, and/or the elongated body 314 can have a consistent type of material (e.g., stainless steel, titanium, or so forth) throughout its different portions. The living hinge can be formed of a same material as the first sidewall 316 and/or the second sidewall 318. Additionally or alternatively, the elongated body 314 can have a different thickness (e.g., greater or lesser) at the top portion 324 than a thickness at other portions to create the living hinge. Moreover, the elongated body 314 can include different materials, such as a first material transitioning to a second, different material via a gradient, and/or different sections formed of different materials, which can also be used to form the living hinge. For instance, the living hinge can be formed of a first material which is a different material than a second material of which the first sidewall 316 and/or the second sidewall 318 are formed. Additionally, the rib fixation device 302 can include a clamping and/or gripping portion(s) extending from the elongated body 314 and/or formed into the elongated body operable to be grabbed by one or more tools, such as forceps. This can include one or more protrusions and/or textured surfaces. With the various features disclosed herein, the rib fixation device 302 can be a multi-functional device in that it can provide stabilization support to the damaged portion 304 of the rib 306 and/or can provide positioning functionality to maintain the biologics-based bone healing agent 104 in place at the damaged portion 304, and/or it can provide a compression onto the biologics-based bone healing agent 104 which further instigates bone growth.

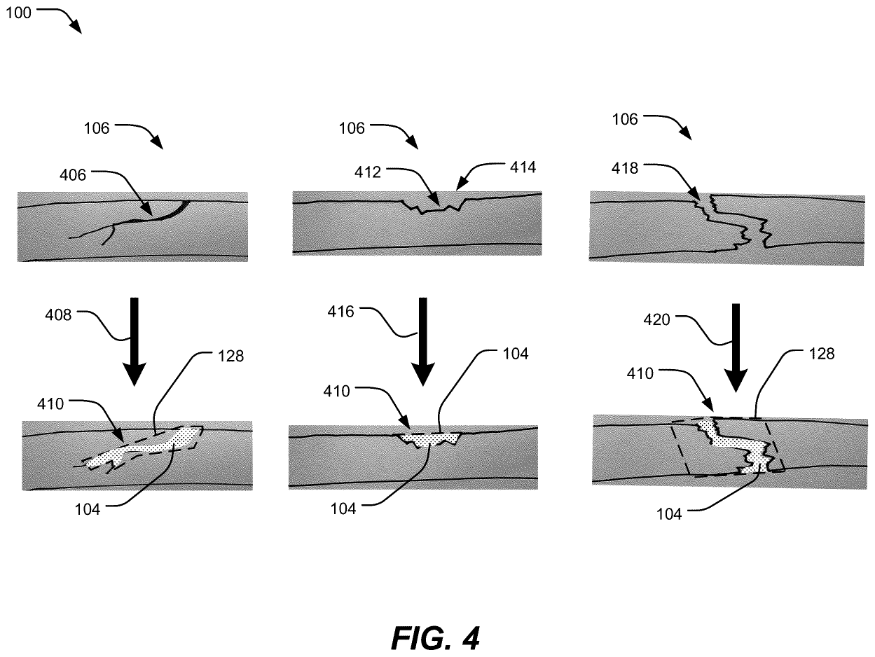

FIG. 4 depicts an example system 100 for treating a thoracic skeletal system 102 with the biologics-based bone healing agent 104. As depicted in FIG. 4, the one or more target area(s) 106 can include a fracture 402 and/or a bone void 404, which can be filled by the biologics-based bone healing agent 104. The system(s) 100 depicted in FIG. 4 can be similar to, identical to, and/or can form at least a portion of the system(s) 100 discussed above regarding FIG. 1.

In some scenarios, the target area 106 can include a first target area 406 which can involve a hairline or linear fracture. A first bone repair procedure 408 can be performed at the first target area 204 to create a regenerative bone-healing structure 410 around the first target area 204. For instance, the first bone repair procedure 408 can include placing the biologics-based bone healing agent 104 in, around, and/or over the hairline fracture. The first bone repair procedure 408 can also include securing (e.g., with adhesives, wires, screws, or so forth, etc.) the thoracic skeletal system fixation hardware 128 (e.g., the fiber mesh 308, the strut 310, the cage 312, and/or the rib fixation device 302) across at least a portion of the hairline fracture and/or positioning a fiber mesh 310 at least partly across the hairline fracture (e.g., beneath and/or over the thoracic skeletal system fixation hardware 128). Additionally or alternatively, the biologics-based bone healing agent 104 can be placed over the thoracic skeletal system fixation hardware 128 and/or can be pushed through openings in the thoracic skeletal system fixation hardware 128 (e.g., and/or openings in the fiber mesh 308). As such, the regenerative bone-healing structure 410 can include a combination of the biologics-based bone healing agent 104 and the thoracic skeletal system fixation hardware 128.

In some examples the target area 106 can include a second target area 412 which can involve a greenstick fracture in which a crack and/or bone void 414 is formed into the outer or inner surface of the bone and/or a portion of bone is missing. A second bone repair procedure 416 can be performed at the second target area 412 to create the regenerative bone-healing structure 410 around the second target area 412. For instance, the second bone repair procedure 416 can include placing the biologics-based bone healing agent 104 in, around, and/or over the greenstick fracture and/or filling the bone void 414. The second bone repair procedure 416 can also include securing (e.g., with adhesives, wires, screws, or so forth, etc.) the thoracic skeletal system fixation hardware 128 (e.g., the fiber mesh 308, the strut 310, the cage 312, and/or the rib fixation device 302) across at least a portion of the bone void 414 to keep the biologics-based bone healing agent 104 in place and/or to provide a compressive force into the second target area 412. Additionally or alternatively, the biologics-based bone healing agent 104 can be placed over the thoracic skeletal system fixation hardware 128 and/or can be pushed through openings in the thoracic skeletal system fixation hardware 128. As such, the regenerative bone-healing structure 410 can include a combination of the biologics-based bone healing agent 104 and the thoracic skeletal system fixation hardware 128.

In some examples, the target area 106 can include a third target area 418 which can involve a transverse, oblique, and/or comminuted fracture. A third bone repair procedure 420 can be performed at the third target area 418 to create the regenerative bone-healing structure 410 around the third target area 418. For instance, the third bone repair procedure 420 can include placing the biologics-based bone healing agent 104 in, around, and/or over the transverse fracture. The third bone repair procedure 420 can also include securing (e.g., with adhesives, wires, screws, or so forth, etc.) the thoracic skeletal system fixation hardware 128 (e.g., the fiber mesh 308, the strut 310, the cage 312, and/or the rib fixation device 302) across at least a portion of the transverse fracture to keep the biologics-based bone healing agent 104 in place. Additionally or alternatively, the biologics-based bone healing agent 104 can be placed over the thoracic skeletal system fixation hardware 128 and/or can be pushed through openings in the fiber mesh 308, the strut 310, the cage 312, and/or the rib fixation device 302. As such, the regenerative bone-healing structure 407 can include a combination of the biologics-based bone healing agent 104 and the thoracic skeletal system fixation hardware 128.

Furthermore, the target area(s) 106 disclosed herein can include a simple fracture, a compound fracture, a greenstick fracture, a hairline fracture, a buckle fracture, an avulsion fracture, a growth plate fracture, a compression fracture, a comminuted fracture, a segmental fracture, a transverse fracture, a spiral fracture, an oblique fracture, and/or any combination thereof. Moreover, a bone repair procedure can include any of the operations of the first bone repair procedure 408, the second bone repair procedure 416, the third bone repair procedure 420, and/or any combination thereof; and/or the bone repair procedure can include any of the thoracic skeletal system fixation hardware 128 and/or biologics-based bone healing agents 104 disclosed herein.

FIG. 5 depicts an example system 100 for treating the thoracic skeletal system 102 with the biologics-based bone healing agent 104. As depicted in FIG. 5 the one or more target area(s) 106 can include one or more vertebra portion(s) 114, for instance, as part of the spinal fusion and stabilization procedure 126. The system(s) 100 depicted in FIG. 5 can be similar to, identical to, and/or can form at least a portion of the system(s) 100 discussed above regarding FIG. 1

In some examples, the target area 106 can include a damaged and/or misaligned thoracic spinal column 502. As such, the thoracic skeletal system fixation hardware 128 can include one or more pedicle screw(s) 134 and/or one or more rod(s) 504 connecting the pedicle screw(s) 134. The pedicle screw(s) 134 can be inserted into the vertebrae 506 being fused together. For instance, a first pedicle screw 508 and/or a second pedicle screw 510 can be inserted into a first vertebra 512, a third pedicle screw 514 and/or a fourth pedicle screw 516 can be inserted into a second vertebra 518 disposed below the first vertebra 512, and so forth for any number of pedicle screws 134 and vertebrae 506. Additionally, a first rod 520 can couple the first pedicle screw 508 to the third pedicle screw 514, a second rod 522 can couple the second pedicle screw 510 to the fourth pedicle screw 516, and so forth.

Furthermore, in some scenarios, the biologics-based bone healing agent 104 can be inserted into the screw holes for the plurality of pedicle screws 134, can coat the rod(s) 504, and/or can be used to fill bone voids 414 in the thoracic spinal column 502. Additionally, various other types of thoracic skeletal system fixation hardware 128 can be used in the spinal fusion and stabilization procedure 126, such as plates, fixation wires 226, struts 310, fiber meshes 308, and/or cages 312. The biologics-based bone healing agent 104 can be used to fill any space between these components of thoracic skeletal system fixation hardware 128 and the bone surface of the thoracic spinal column 502. Moreover, the biologics-based bone healing agent 104 can fill holes, openings, and/or gaps in the thoracic skeletal system fixation hardware 128 itself (e.g., screw holes, corners, perforations, etc.). The biologics-based bone healing agent can also fill bone voids 414 and/or other bone fractures in the vertebrae 506 of the thoracic spinal column 502, such as the hairline or linear fracture of the first target area 406, the bone void 414 of the second target area 412, the transverse, oblique, and/or comminuted fracture of the third target area 418, and/or any combinations thereof.

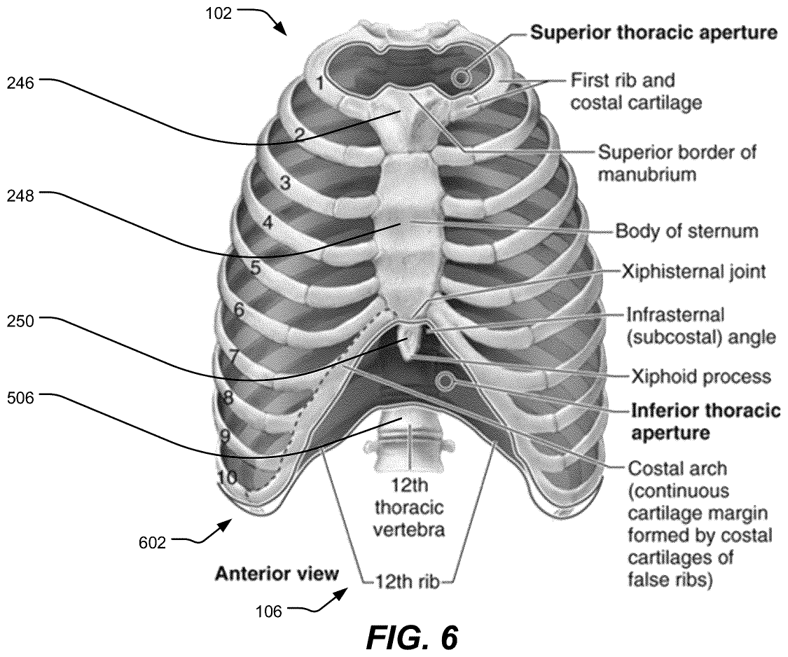

FIG. 6 depicts an example system 100 for treating the thoracic skeletal system 102 with the biologics-based bone healing agent 104 inserted into one or more target area(s) 106. Any of the techniques discussed above regarding FIGS. 1-5 can be used to heal any portion of the thoracic skeletal system 102. For instance, the target area(s) 106 can include one or more of a superior border of the manubrium 246, the sternum body 248, a xiphisternal joint, an infrasternal (subcostal) angle, the xiphoid process 250, an edge of an inferior thoracic aperture, an edge of a superior thoracic aperture, any of ribs 1-12, a thoracic vertebra 506, combinations thereof, and/or any other portion of the thoracic cage 602. Moreover, the system 100 can include any combination of target areas 106. For instance, a thoracic skeletal treatment system can include performing any or all of the sternal closure and/or reconstruction procedure 122, the rib fixation and healing procedure 124, and/or the spinal fusion and stabilization procedure 126.

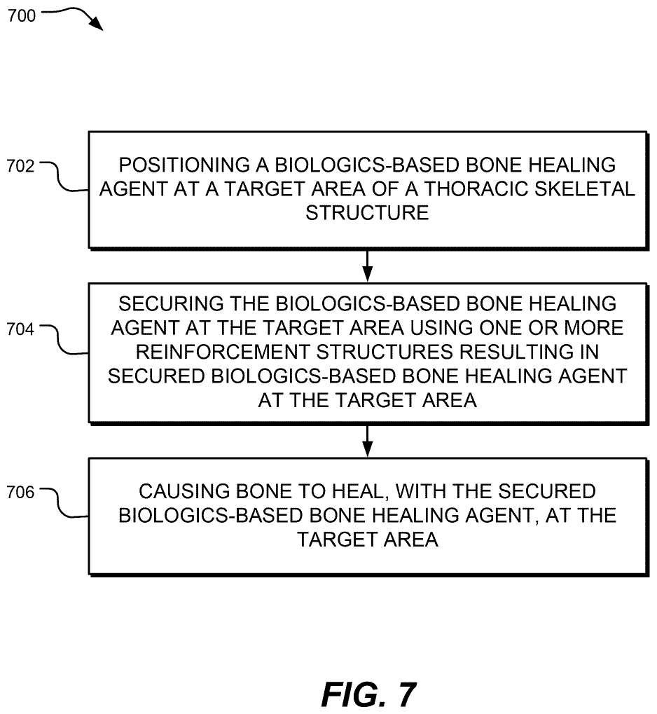

FIG. 7 depicts example method(s) 700 for treating the thoracic skeletal system 102 with the biologics-based bone healing agent 104. The method(s) 700 can be performed by any of the system(s) 100 discussed herein regarding FIGS. 1-6.

In some examples, at operation 702, the method 700 can position a biologics-based bone healing agent at a target area of a thoracic skeletal structure. At operation 704, the method 700 can secure the biologics-based bone healing agent at the target area using one or more reinforcement structures resulting in secured biologics-based bone healing agent at the target area. At operation 706, the method 700 can cause bone to heal, with the secured biologics-based bone healing agent, at the target area.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any of the features or components of one example can be combined with any feature or components of any other example. These and other variations, modifications, additions, and improvements may fall within the scope of the disclosure as defined in the claims that follow.

What is claimed is:

1. A method of thoracic skeletal structure treatment, the method comprising:
positioning a biologics-based bone healing agent at a target area of a thoracic skeletal structure;
securing the biologics-based bone healing agent at the target area using one or more reinforcement structures resulting in secured biologics-based bone healing agent at the target area; and
causing bone to heal, with the secured biologics-based bone healing agent, at the target area.

2. The method of claim 1,
wherein,
the target area of the thoracic skeletal structure includes at least one of:
a thoracic cage portion,
a rib portion,
a sternum portion,
a clavicle portion,
a cervical vertebrae portion,
a thoracic vertebrae portion,
a lumbar vertebrae portion, or
a scapula portion.

3. The method of claim 2,
wherein,
the target area includes at least one of:
a damaged bone tissue,
a degenerated bone tissue,
a demineralized bone tissue,
a genetically deformed bone tissue,
an idiopathically deformed bone tissue,
an infected bone tissue,
an iatrogenically destabilized bone tissue, or
a traumatized bone tissue.

4. The method of claim 1,
wherein,
the biologics-based bone healing agent has a bone growth factor including at least one of exosomes or bone morphogenetic proteins (BPM)s.

5. The method of claim 4,
wherein,
the biologics-based bone healing agent has a form factor of at least one of:
a bone graft,
a synthetic graft,
a putty,
a gel,
a bioglass,
a powder,
an injectable gel or liquid, or
a collagen matrix.

6. The method of claim 1, further comprising:
reducing bleeding at the target area using the biologics-based bone healing agent.

7. The method of claim 1,
wherein,
the target area includes a first sternal section separated from a second sternal section during a sternotomy, and
the causing of the bone to heal at the target area includes performing a sternal closure and reconstruction for the first sternal section and the second sternal section.

8. The method of claim 1,
wherein,
the target area includes a rib fracture, and
the causing of the bone to heal at the target area includes a rib fixation and healing procedure at the rib fracture.

9. The method of claim 1,
wherein,
the target area includes a thoracic spinal vertebra, and
the causing of the bone to heal at the target area includes a spinal fusion and stabilization procedure at the thoracic spinal vertebra.

10. The method of claim 1,
wherein,
the target area includes a bone void, and
the positioning of the biologics-based bone healing agent includes filling the bone void with a regenerative bone putty.

11. The method of claim 1, further comprising positioning a thoracic skeletal system stabilization hardware device around the target area, and the positioning of the biologics-based bone healing agent includes filling a gap between the thoracic skeletal system stabilization hardware device and bone tissue with a regenerative bone putty.

12. The method of claim 1, further comprising positioning a thoracic skeletal system stabilization hardware device around the target area, and the positioning of the biologics-based bone healing agent includes filling a screw hole of the thoracic skeletal system stabilization hardware device with a regenerative bone putty.

13. A method of thoracic skeletal structure treatment, the method comprising:
positioning a biologics-based bone healing agent at a target area located at one or more of: a rib, a sternum, or a thoracic vertebra;
securing the biologics-based bone healing agent at the target area resulting in secured biologics-based bone healing agent at the target area; and
causing a damaged portion of bone to heal, with the secured biologics-based bone healing agent, at the target area.

14. The method of claim 13,
wherein,
the biologics-based bone healing agent includes a sternal implant disposed between two sternal bone sections, and
the securing of the biologics-based bone healing agent includes compressing the two sternal bone sections together with one or more wires, the one or more wires include one or more force dispersion supplements which increase a surface area of the one or more wires against the two sternal bone sections.

15. The method of claim 13,
wherein,
the target area includes a portion of a thoracic spinal column stabilized with stabilization hardware including at least one of: a pedicle screw, a plate, or a wire, and
the positioning of the biologics-based bone healing agent at the target area includes filling a space around the stabilization hardware.

16. The method of claim 13,
wherein,
the positioning of the biologics-based bone healing agent at the target area includes filling a void with the biologics-based bone healing agent resulting in a filled void, and
the securing of the biologics-based bone healing agent at the target area includes creating a rigid environment under compression at the filled void which instigates bone growth.

17. A method of thoracic skeletal structure treatment, the method comprising:
positioning a biologics-based bone healing agent at a target area of a thoracic skeletal structure by filling a bone void, resulting in a filled bone void;
securing the biologics-based bone healing agent at the target area resulting in secured biologics-based bone healing agent at the target area; and
causing bone to heal, with the secured biologics-based bone healing agent, at the target area.

18. The method of claim 17,
wherein,
the bone void is located on at least one of: a rib, a sternum, or a vertebra.

19. The method of claim 17,
wherein,
the biologics-based bone healing agent includes a liquid, gel, or putty, and
securing the biologics-based bone healing agent includes solidifying the liquid, the gel, or the putty.

20. The method of claim 17,
wherein,
the securing of the biologics-based bone healing agent includes mechanically stabilizing the target area and applying compression on the filled bone void, and
the causing of the bone to heal includes instigating bone growth responsive to the compression on the filled bone void.
